(19) United States
(12) Patent Application Publication
(10) Pub. No.: US 2026/0157639 A1
(43) Pub. Date: Jun. 11, 2026

(54) PUTATIVE ENERGY FIELD ANALYSIS USING NON-THERMAL PLASMA ARRAY

(71) Applicant: Iolera Holdings PTE. LTD, Parkview Square (SG)

(72) Inventors: Bradley N. ECKERT, Chandler, AZ (US); Bryon K. Eckert, Chandler, AZ (US); Huan Truong, Chandler, AZ (US)

(21) Appl. No.: 19/178,762

(22) Filed: Apr. 14, 2025

Related U.S. Application Data
(63) Continuation of application No. 17/609,333, now Pat. No. 12,274,533.
(60) Provisional application No. 62/843,978, filed on May 6, 2019.

(51) Int. Cl. A61B 5/0205; A61B 5/0245; A61B 5/053

(52) U.S. Cl. CPC A61B 5/0205; A61B 5/0245; A61B 5/053

(57) ABSTRACT

A device for detecting and analyzing vital fields includes: a dielectric barrier discharge array fabricated on a thin substrate with low dielectric loss; an air permeable sheet for electrical insulation from the skin; a transformer for generating sufficient AC voltage to cause air breakdown in the array; a signal transformer and bypass capacitor for isolating the radio frequency current from the plasma discharge; circuitry for amplification and narrow-band spectrum analysis of the plasma discharge current. The amplified signal from the plasma discharge current is gated to include only signal from the part of the drive waveform where plasma discharge predominantly occurs. Frequency converters reduce the complexity of narrow-band spectrum analysis; spectrum analysis is done by Fast Fourier Transform analysis of the frequency converter outputs. Different FFT results are compared and analyzed to aid the user with the correct array placement on the body, and the detection of medical conditions.

### FIELD OF INVENTION

This invention relates to pattern analysis. In particular it relates particularly to methods and devices for detecting and analyzing energy fields emitted by organisms.

### BACKGROUND

All live organisms emit energy fields, referred to herein as vital fields, which are characterized by the organic processes that produce or modify them. There is a significant amount of skepticism surrounding vital fields because no known scientific instruments can detect them. The inability to detect, measure, and describe the energy in a vital field is a problem that inhibits human understanding of biological interactions with the environment.

A wave in an energy field is considered to be composed of electric, magnetic, gravitational and temporal components. The electric, magnetic, and gravitational components are orthogonal to each other. In an electromagnetic wave, the gravitational and temporal components have a static value, and the electric and magnetic components vary inversely. In this context, a static temporal component equates to time moving forward at a constant rate. In contrast, a vital wave is theorized to contain static electric and magnetic components and dynamic temporal and gravitational components. Such a wave is essentially a longitudinal or compression wave in the space-time fabric, but is often referred to as a torsion wave. Because vital waves do not have a dynamic magnetic component, they do not induce a current in a conductor. Most known devices rely on such induction and are therefore unable to reliably detect the presence of vital waves or measure or describe them scientifically.

Kirlian photography, discovered in the early 20th century, can be considered one of the earliest means of analyzing vital fields. Kirlian photography works by driving a photographic plate at high voltage, with a biological specimen resting on the plate. The resulting image left on the film is consistent with the corona discharge pattern of the specimen. Live specimens tend to show a shimmering coronal effect, whereas dead specimens and inanimate objects exhibit a more uniform pattern. The difference is attributed to the live specimen having at least one vital field. It should be noted, however, that Kirlian photography as an indicator of vital fields has been met with skepticism, with the results explained away as errors in the experimental process.

Plasmas are a fourth possible state of matter, the others being solid, liquid, and gas. Plasmas are formed when a gas is subjected to high stresses that create a mixture of neutral atoms, positively charged atomic and molecular ions, and freed electrons. Non-thermal plasmas occur when the electrons are much more energetic than the neutral or positively charged particles. The application of non-thermal plasma in medicine has recently been a fruitful field of research. The beneficial health effects of non-thermal plasma applications in living organisms are often attributed to reactive oxygen or nitrogen species. Closer investigation of the health effects imparted by an array of micro-plasmas placed near the skin shows energetic effects that are not explained by conventional science. If these energetic effects can be electronically analyzed in real time, the information can be used to provide feedback to the user to show the optimum application of plasma to the body, the progress of healing, and the amount of pain.

### SUMMARY

The present device is a dielectric barrier discharge array fabricated on a thin substrate with low dielectric loss. This can be FR-4, PEEK, a cyclic olefin fiberglass laminate or other similar low loss material. The oxygen plasma will erode the polymer over time, but the array lifetime can be extended with a thin film of oxide, typically aluminum oxide deposited by atomic layer deposition.

An applied AC voltage causes breakdown of the surrounding air in the sub-nanosecond range. The array is typically covered by an air permeable, electrically insulating sheet and placed next to the skin. The velocity of the plasma away from the electrodes is inversely proportional to the size of the plasma discharge. In the case of the micro-plasma, it is sufficiently high to cause relativistic effects. This converts the torsion waves of the vital field emanating from the body to electromagnetic waves that are conducted through the plasma array.

The array of plasma plumes provides a large number of mutually coupled areas of population inversion. This is caused by the collapsing non-linear magnetic field during the plasma discharge. The polar nature of the oxygen molecule causes a large number of hyperfine resonant frequencies from 53 GHz to 2.5 THz and beyond. At these frequencies, amplification by stimulated emission can take place.

Because each plasma plume discharges at a slightly different time, spontaneous emission from the array will manifest as broadband noise. However, a biological specimen will generate a large number of modulated carriers close to the hyperfine resonant frequencies of oxygen. These carriers are not detectable with normal millimeter-wave electronics, since there is no dynamic magnetic component in the torsion waves that comprise the vital energy field.

When the array is placed close to the skin, the plasma plumes convert this energy to electromagnetic waves. This energy can be detected by millimeter-wave electronics, but the cost is very high. This approach is not suited to a consumer device intended for broad usage. However, the conversion process between torsion wave vital energy and electromagnetic energy through plasma interaction is a non-linear process. This creates mixing products between the carriers, some of which can be analyzed in the VHF and UHF frequency ranges. To help reject radio signals, these signals are analyzed by narrow-band spectrum analysis.

### BRIEF DESCRIPTION OF DRAWINGS

FIG. 1 is a schematic diagram of the present device.

FIG. 2 is a block diagram of the signal analysis in the preferred embodiment.

### DETAILED DESCRIPTION

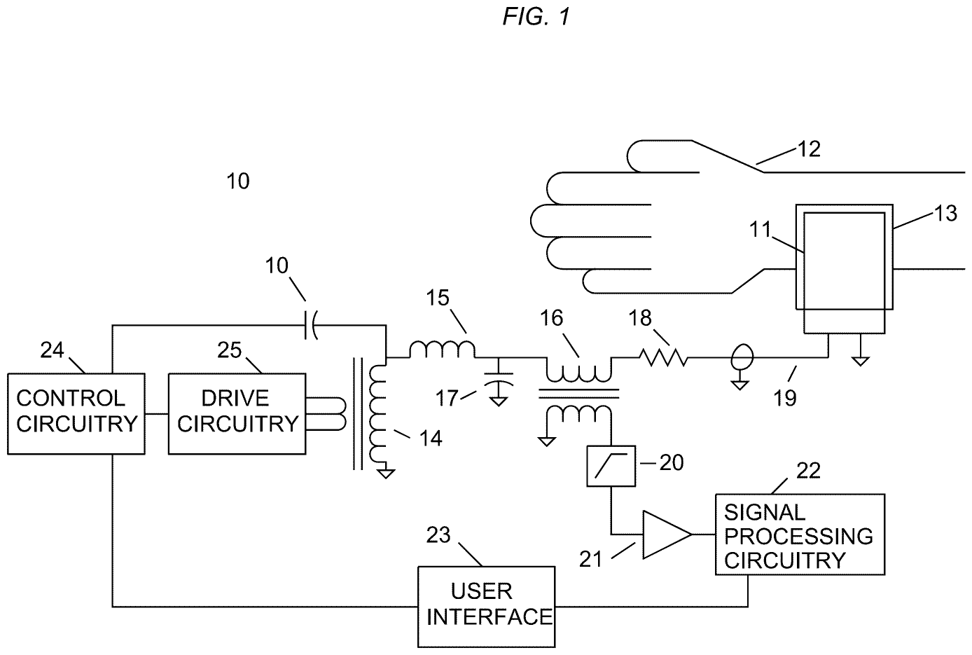

FIG. 1 illustrates the presently described systems and a example method of use. The system 10 may generate an array of non-thermal plasma plumes for plasma medicine applications. An array 11 electrically insulated by an air-permeable sheet or pouch 13 may be placed next to the skin 12 of a living organism. The array 11 is driven by high voltage transformer 14, through high frequency filter inductor 15, signal transformer 16, resistor 18 and optional coaxial cable 19. A combination of bypass capacitor 17 and signal transformer 16 may be used to detect high frequency current from the array 11 under conditions of air discharge. Resistor 18 is used to reduce signal reflections to allow a flatter frequency response.

An output of signal transformer 16 is filtered by high pass filter 20 to remove the array drive frequency component, which may typically be a value between 50 kHz and 150 kHz. Because of the array capacitance, the amplitude of the reverse power decreases with increasing frequency. The electric current frequency range is typically 10 MHz to 840 MHz in the high-frequency array. Low Noise Amplifier 21 may be used to minimize the system noise floor. This is connected to signal processing circuitry 22 and user interface 23. In a preferred embodiment the plasma discharges only occur in a defined phase window of the driving waveform. The ratio between the plasma current noise floor and the system thermal noise floor is maximized by gating the signal with a defined duty cycle and phase offset.

The drive circuitry 25 provides a primary drive for high voltage transformer 14. The control circuitry 24 provides power control and fault protection in case of array failure. In an example embodiment, the resonant frequency of the transformer and array combination is measured by the controller in a calibration routine. This may allow a faster shutdown in case of array failure or detection of array contamination. An alternative embodiment may use a self-oscillating transformer primary drive.

In this configuration, a low inductance is needed to overcome the capacitance of the array in order to drive it at high frequency. Due to the relatively small number of turns in the transformer, which carry the induced magnetic flux, the flux density in the ferrite core will be high. A high flux density causes high power dissipation in the core. In some embodiments this may be mitigated by modulating the AC voltage to reduce the average power dissipation, for example at a duty cycle of 30%.

In one embodiment, the modulation frequency may typically be set between 10 Hz and 5 kHz. Modulation frequency-dependent energetic effects have been demonstrated in-vitro and in-vivo by numerous researchers in plasma medicine. In a preferred embodiment, the modulation dictates a relatively narrow analysis bandwidth of about 11 kHz. In a preferred embodiment, the signal processing circuitry 22, user interface 23 and control circuitry 24 are implemented in an ASIC.

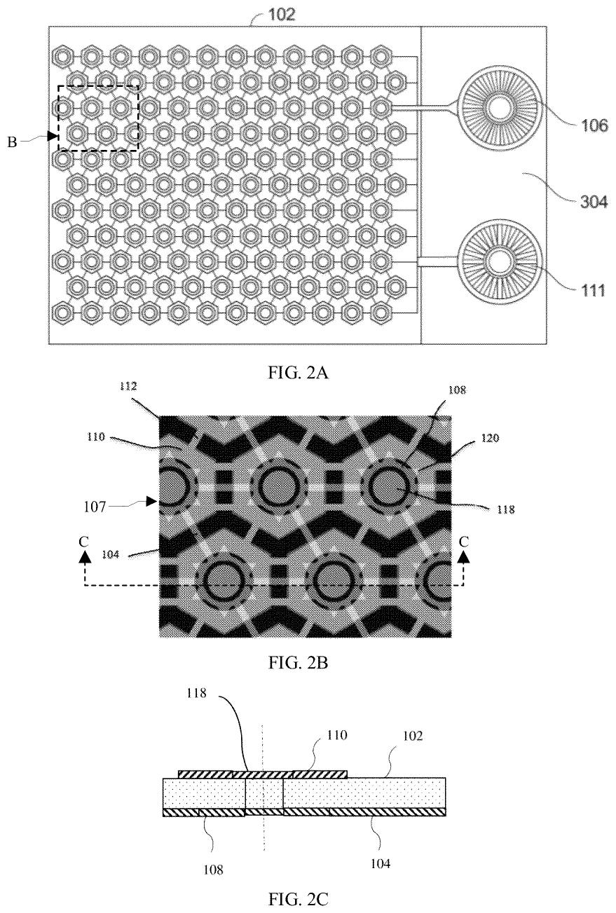

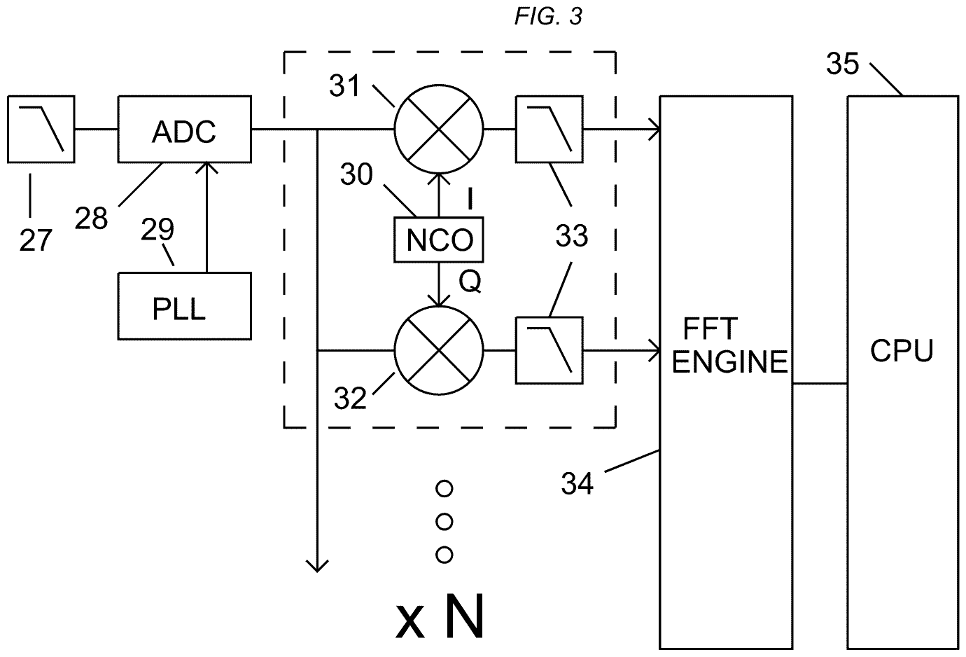

The signal analysis approach in the preferred embodiment of device 10 is shown in FIG. 2. In a typical embodiment, the high speed analog-to-digital converter (ADC) is implemented as a set of lower speed ADCs with a set of high speed sample-and-hold circuits with fixed phase offset between samples.

A set of eight ADCs with Successive Approximation Array (SAR) architecture clocked at one eighth of the input clock rate will allow low power consumption and smaller die area relative to a higher performance solution.

For digital down-conversion, the ASIC digital logic is clocked in the UHF frequency range, such as 460 MHz in an example embodiment, using parallel multipliers to accommodate the ADC sample clock rate of 1840 MHz. The signal processing circuitry begins with a low pass analog filter 27 and high speed ADC 28.

In order to minimize the probability of a clock harmonic blocking the desired signal, the ASIC clock input may be half of this frequency. Using a higher frequency is no advantage as the minimum clock rate is set by the individual SAR ADC clocks. The clock is preferably voltage tuned to mitigate the problem of desired signals being masked by spurious signals within the digital receiver.

In a typical embodiment, the ASIC input clock frequency is approximately 230 MHz to support the parallel ADC interface. Phase-locked loop 29 multiplies the 230 MHz reference by a factor of 8 to clock an ADC at 1840 MHz. This supports analysis frequencies past 840 MHz.

In a digital down-converter, a Numerically Controlled Oscillator (NCO) 30 is also clocked at 1840 MHz. This drives an In-phase (I) multiplier 31 and Quadrature (Q) multiplier 32. A decimation occurs in low pass filters 33, typically implemented as a combination of cascaded integrator-comb (CIC) and finite impulse response (FIR) filters. Like an ADC, a high speed digital section may be implemented in one or more sections operated in parallel and clocked at a lower speed.

An ADC output may be connected to multiple digital down converters with a typical decimation factor of 65520, resulting in a final sample rate of 28 kHz. This process would yield an approximate upper bandwidth limit of about 10.5 kHz. In a preferred embodiment, frequency stability will be within 0.5 ppm, or 420 Hz at the highest frequency. This permits analysis within the highest frequency modulation side bands.

In a preferred embodiment, the outputs of filters 33 are analyzed in an 8192 point fast Fourier transform (FFT), preferably by dedicated FFT hardware 34 and by a CPU 35. The complex FFT outputs are added or subtracted to measure signals on a particular side of the NCO carrier. Since the plasma discharges only occur in a defined phase window of the driving waveform, much of the FFT input will be set to zero. The 8192 point FFT will require 280 ms to collect data.

In a preferred embodiment, the ASIC would typically be implemented in a 28 nm low power CMOS process. This would allow efficient power usage for the ADC and high speed digital circuitry, and permit 16 digital down converters within a total die size of about 2.5 mm square.

A relatively large number of digital down converters will permit flexible tuning and analysis, examining multiple side bands on a small number of carriers, or fewer side bands on a large number of carriers.

In an alternative embodiment, a digital down converter uses a small decimation factor along with a large FFT to search for signals in a large frequency range. In this case, the ADC clock operates at three times the 10G ethernet reference clock, or 1933.594 MHz. This minimizes spurious signals as the ethernet clock is running during data capture.

The down converter output is transferred to a PC over a QSFP+ connection (40 Gbps) and analyzed by a GPU card with a large memory bandwidth, such as an Nvidia Tesla K-40.

The GPU card runs a set of 12 FFTs of typical size 2\hat{ }25, combined with a window function for the FFT input and a sort function for the FFT output. This output may be used to find signals for various medical conditions or biological specimens.

Each torsion wave signal is expected to be modulated in the audio frequency range, so the maser inter-modulation signals are expected to contain mixing products of these modulation side bands. These signals may contain further mixing products with the plasma drive frequency and the plasma modulation frequency.

The outputs of each complex FFT will contain amplitude and phase information. The frequency, phase and amplitudes of the relevant FFT outputs may be analyzed to determine correlation to medical conditions, acupuncture meridians, pain and other parameters. Since a relatively small number of signal peaks are needed for data analysis, the data can be analyzed on the ASIC, or streamed over a USB or wireless connection.

In certain configurations, the plasma array may also act as a radio antenna. The system in the present disclosure includes a plasma feedback radio receiver device. When the plasma array is operated away from the body of a living organism the radio receiver device is able to collect data about the ambient environment. Collected results may be stored in a database connected to the system. Data from the database may be used to calibrate the plasma array for use with living organism. The stored results in the database may be used to differentiate ambient environmental radio signals from biofield signals generated by a living organism when the plasma array is operated next to the body.

What is claimed is:

1. A device for detecting and analyzing vital fields, the device comprising:
a dielectric barrier discharge array fabricated on a thin substrate with low dielectric loss;
an air permeable sheet for electrical insulation from the skin;
a transformer for generating sufficient AC voltage to cause air breakdown in the array;
a signal transformer and bypass capacitor for isolating the radio frequency current from the plasma discharge;
circuitry for amplification and narrow-band spectrum analysis of the plasma discharge current wherein:
the amplified signal from the plasma discharge current is gated to include only signal from the part of the drive waveform where plasma discharge predominantly occurs;
a multiplicity of frequency converters are used to reduce the complexity of narrow-band spectrum analysis;
spectrum analysis is done by Fast Fourier Transform (FFT) analysis of the frequency converter outputs;
the different FFT results are compared and analyzed to aid the user with the correct array placement on the body, and the detection of medical conditions.

2. The device of claim 1 wherein the frequency converters are implemented as analog circuitry.

3. The device of claim 1 wherein the amplified signal is digitized by a high speed analog to digital converter and the frequency converters are implemented as digital down converters and decimating filters.

4. The device of claim 3 wherein a small decimation digital down converter, or no down converter, is combined with a very long FFT to search for signals over a large range.

5. The device of claim 1 wherein the array is connected to the signal transformer through a length of coaxial cable.

6. The device of claim 4 wherein a series resistor is added to reduce signal reflections on the coaxial cable.

7. The device of claim 1 wherein the drive control for the high voltage transformer is integrated into an Application Specific Integrated Circuit (ASIC), along with the narrow-band spectrum analysis circuitry and an associated Central Processing Unit (CPU) for real-time analysis.

8. The device of claim 7 wherein the ASIC is connected to a Bluetooth transceiver for the purpose of using a smart phone as a secondary user interface.

9. The device of claim 7 wherein the ASIC consists of narrow-band spectrum analysis circuitry and an associated Central Processing Unit (CPU) for real-time analysis, and the plasma control is performed by a separate micro-controller.

10. The device of claim 1 wherein the plasma is modulated by turning it on and off at a set frequency and duty cycle.

11. The device of claim 10 wherein the plasma modulation frequency is set to address the treatment of a specific physical condition.

12. The device of claim 10 wherein reverse power generated by the plasma is measured and used by the controller to find an optimum modulation frequency.

13. The device of claim 10 wherein the measured reverse power is used to provide feedback to the user.

14. The device of claim 10 wherein radio signals in the VHF and UHF frequency ranges, generated by plasma interaction with the body, are detected for the purpose of adjusting the modulation frequency.
